(19) United States
(12) Patent Application Publication
(10) Pub. No.: US 2015/0005787 A1
(43) Pub. Date: Jan. 1, 2015

(54) SLICK EXTRACTOR

(71) Applicant: WINNIFRED AHMED MOHAMMED-HARLAND, Lilburn, GA (US)

(72) Inventor: WINNIFRED AHMED MOHAMMED-HARLAND, Lilburn, GA (US)

(21) Appl. No.: 13/895,293

(22) Filed: Jul. 1, 2013

(51) Int. Cl. A61B 17/50; A61M 1/08

(52) U.S. Cl. CPC A61B 17/50; A61M 1/08; A61B 2017/00747
USPC 606/131

(57) ABSTRACT

Slick Extractor is a hand held rechargeable extractor with a suction power designed to extract oil and other impurities like dirt and dust from the pores of the face and skin. This innovation, use the power of a mini motto to practically suck out dirt and oil from the pores of the skin without chemical and, without leaving it dry. Slick Extractor runs on a power rechargeable mini motto, powerful enough for suction, yet gentle enough to be used on the surface of the facial skin. Its mini size makes it easy to use on all part of the skin.

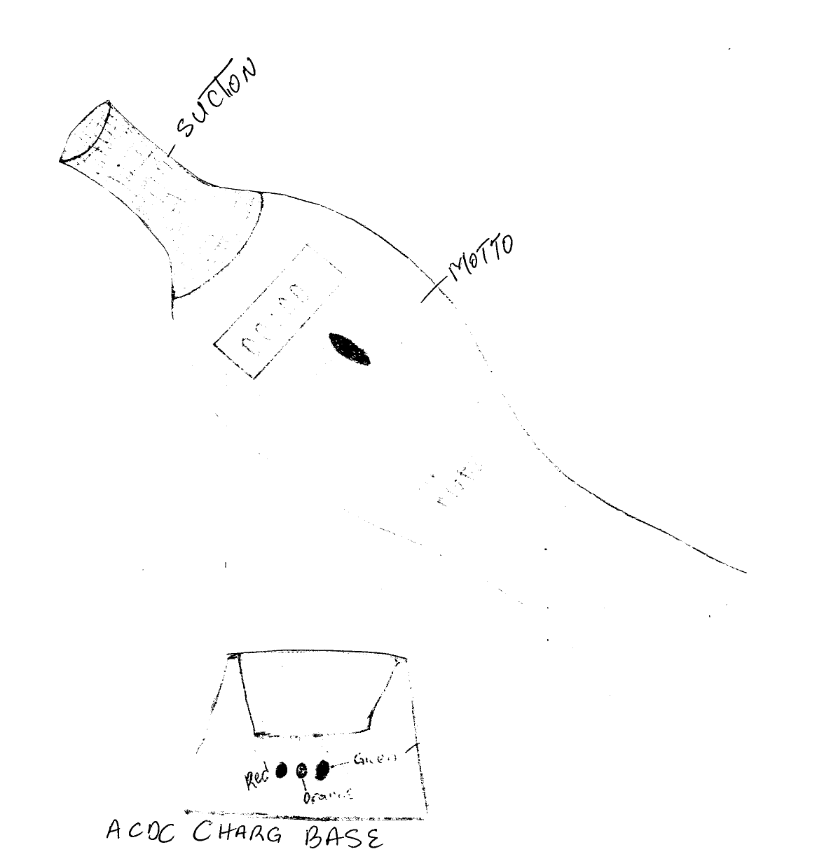

Slick Extractor by Slick Novations is a hand-held rechargeable extractor designed to extract oil and other impurities from facial pores or any part of the body.

With the power of a hand-held vacuum designed to practically vacuum or suck oil and other impurities from the surface of the skin by a flick of a button.

1. The innovative claim of Slick Extractor is its suction cup and its hand held mini running motto that is powerful enough for suction but, gentle enough to be used on the surface of the facial skin. Slick Extractor is power rechargeable hand held mini motto extractor, designed to extract oil, dirt and dust from the pores of the skin. Slick Extractor is the only known process which uses a hand held mini suction motto to extract oil and impurities from the pores of the surface of the facial skin without the application of chemicals. The use of this mini device will help minimize facial pores by keeping them free from the accumulation of oil, dust and other impurities. This will help keep the pores free from bacteria, thereby; preventing the formation of pimples, blackheads and other skin irritation caused by excessive facial oil.
